(19) United States
(12) Patent Application Publication
(10) Pub. No.: US 2007/0244442 A1
(43) Pub. Date: Oct. 18, 2007

(54) Device for Ophthalmic Drug Delivery

(76) Inventor: Masood A. Chowhan, Arlington, TX (US)

Correspondence Address: ALCON, IP LEGAL, TB4-8, 6201 SOUTH FREEWAY, FORT WORTH, TX, 76134, US

(21) Appl. No.: 11/749,543

(22) Filed: May 16, 2007

Related U.S. Application Data
(63) Continuation of application No. PCT/US05/45459, filed on Dec. 15, 2005.
(60) Provisional application No. 60/638,775, filed on Dec. 22, 2004.

(51) Int. Cl. A61M 5/19

(52) U.S. Cl. 604/191

(57) ABSTRACT

An ophthalmic drug delivery device having two actuation assemblies for dispensing incompatible dosage forms and facilitating the prevention of dosage form reflux.

This application is a continuation of PCT/US2005/045459 filed Dec. 15, 2005 entitled “Device for Ophthalmic Drug Delivery,” which claims priority from U.S. Provisional Application No. 60/638,775 filed Dec. 22, 2004.

### FIELD OF THE INVENTION

The present invention generally pertains to a device for ophthalmic drug delivery. More particularly, but not by way of limitation, the present invention pertains to such a device for posterior segment ophthalmic drug delivery.

### DESCRIPTION OF THE RELATED ART

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

ARMD is the leading cause of blindness in the elderly of developed countries. ARMD attacks the center of vision and blurs it, making reading, driving, and other detailed tasks difficult or impossible. About 200,000 new cases of ARMD occur each year in the United States alone. Current estimates reveal that approximately forty percent of the population over age 75, and approximately twenty percent of the population over age 60, suffer from some degree of macular degeneration. “Wet” ARMD is the type of ARMD that most often causes blindness. In wet ARMD, newly formed choroidal blood vessels (CNV) leak fluid and cause progressive damage to the retina.

In the particular case of CNV in ARMD, three main methods of treatment are currently being developed, (a) photocoagulation, (b) photodynamic therapy, and (c) the use of angiogenesis inhibitors. Photocoagulation is the most common treatment modality for CNV. However, photocoagulation can be harmful to the retina and is impractical when the CNV is near the fovea. Furthermore, over time, photocoagulation often results in recurrent CNV. Photodynamic therapy is a relatively new technology. The long-term efficacy of photodynamic therapy to treat ARMD is still largely unknown. Oral or parenteral (non-ocular) administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required.

Various needles and cannulae have been used to deliver drugs to the back of the eye, external to the globe. Examples of such needles and cannulae are disclosed in U.S. Pat. No. 6,413,245 and the references cited therein. U.S. Pat. No. 6,413,245 discloses preferred cannulae for sub-Tenon, juxtascleral delivery of a drug depot to the posterior segment of a human eye and is incorporated herein by reference. These preferred cannulae have a distal portion with a radius of curvature substantially equal to the radius of curvature of the globe of the human eye. When these cannulae are used to create such a drug depot, drug reflux may sometimes occur during or immediately after administration.

A need remains in the field of ophthalmology for improved devices for the administration of an ophthalmic drug, especially to the posterior segment of the eye. Improved devices are also needed to minimize or prevent drug reflux as described above, and to facilitate drug depot placement. These improved devices should be safe for the patient, should be easy for the physician to use, and should improve the efficacy of drug administration.

### SUMMARY OF THE INVENTION

The present invention is an ophthalmic drug delivery device including a body having a plunger chamber, a first actuation chamber, and a second actuation chamber. A plunger assembly having a first sealing member is slidably disposed within the plunger chamber. The device includes a first actuation assembly having a first contact member disposed in the plunger chamber, a second sealing member slidably disposed in the first actuation chamber, and a spring member disposed between the first sealing member and the first contact member. The device also includes a second actuation assembly having a second contact member disposed in the plunger chamber and a third sealing member slidably disposed in the second actuation chamber. A cannula is fluidly coupled to the first actuation chamber and the second actuation chamber.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a front, sectional, schematic view of a drug delivery device according to a preferred embodiment of the present invention with the plunger assembly in a fully undepressed position;

FIG. 2 is a fragmentary, front, sectional, schematic view of the device of FIG. 1 with the plunger assembly in a partially depressed position;

FIG. 3 is a fragmentary, front, sectional, schematic view of the device of FIG. 1 with the plunger assembly in a fully depressed position; and

FIG. 4 is a front, sectional, schematic view of a drug delivery device according to a second preferred embodiment of the present invention with the plunger assembly in a fully undepressed position.

### DETAILED DESCRIPTION OF THE PREFERRED EMBODIMENTS

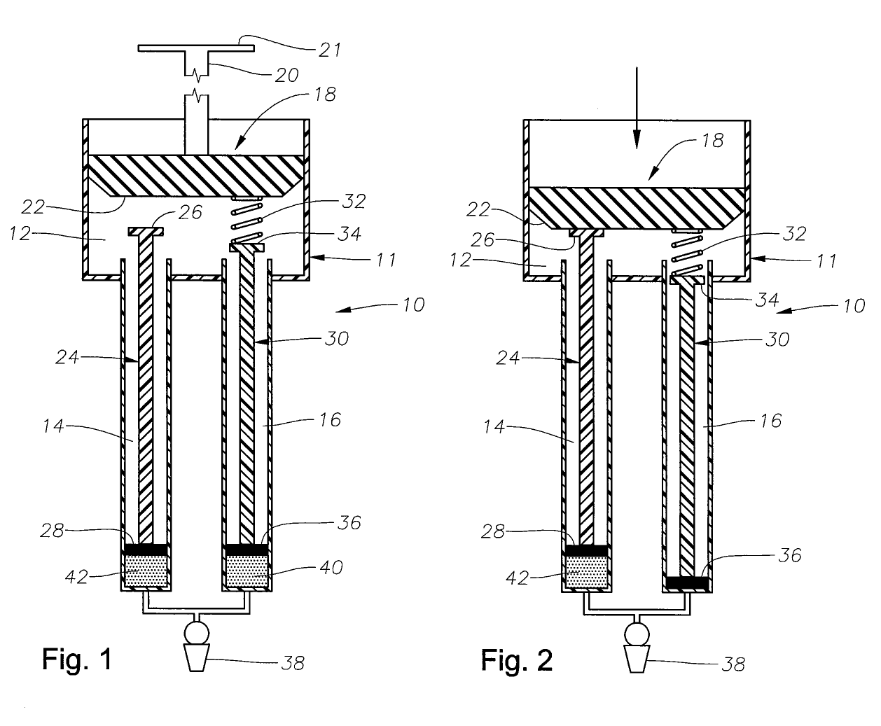

The preferred embodiments of the present invention and their advantages are best understood by referring to FIGS. 1-4 of the drawings, like numerals being used for like and corresponding parts of the various drawings.

As shown in FIG. 1, drug delivery device 10 preferably includes a body 11 having a plunger chamber 12, an actuation chamber 14, and an actuation chamber 16; a plunger assembly 18 having a handle 20 and a sealing member 22; an actuation assembly 24 having a contact member 26 and a sealing member 28; an actuation assembly 30 having a spring member 32, a contact member 34, and a sealing member 36; and a cannula 38 fluidly coupled to both actuation chamber 14 and actuation chamber 16. Device 10 is preferably sized so as to comfortably fit within a physician's hand.

Sealing member 22 is in slidable, fluid tight engagement with the interior surface of plunger chamber 12. Spring member 32 is preferably coupled to sealing member 22 on a first end and contact member 36 on a second end. Sealing member 28 is in slidable, fluid tight engagement with the interior surface of actuation chamber 14. Sealing member 36 is in slidable, fluid tight engagement with the interior surface of actuation chamber 16. Cannula 38 may be any conventional blunt-tip cannula or sharp-tip needle suitable for ophthalmic drug delivery. Preferred cannulae for cannula 38 for use in sub-Tenon, juxtascleral delivery of a drug depot to the posterior segment of a human eye are disclosed in U.S. Pat. No. 6,413,245.

A dosage form 40 is disposed within actuation chamber 16 between sealing member 36 and cannula 38. A dosage form 42 is disposed within actuation chamber 14 between sealing member 28 and cannula 38. Device 10 is preferably packaged with dosage forms 40 and 42 preloaded. Alternatively, dosage forms 40 and 42 may be loaded by the user prior to administration.

Dosage forms 40 and 42 may be any dosage form containing a drug or pharmaceutically active agent. Dosage forms 40 and 42 may be in liquid, semi-solid, or solid form. For example, dosage forms 40 and 42 may be a solution, a suspension, an emulsion, an ointment, a gel forming solution, a gel, a bioerodable polymer, a non-bioerodable polymer, or a powder. Preferably, dosage forms 40 and 42 include any ophthalmically acceptable pharmaceutically active agent. Examples of pharmaceutically active agents suitable for dosage forms 40 and 42 are disclosed in U.S. Pat. No. 6,416,777, which is incorporated herein by reference. One preferred pharmaceutically active agent is angiostatic steroids for the prevention or treatment of diseases or conditions of the posterior segment of the eye, including, without limitation, ARMD, CNV, retinopathies, retinitis, uveitis, macular edema, and glaucoma. Such angiostatic steroids are more fully disclosed in U.S. Pat. Nos. 5,679,666 and 5,770,592, which are incorporated herein by reference. Preferred ones of such angiostatic steroids include 4,9(11)-Pregnadien-17α,21-diol-3,20-dione and 4,9(11)-Pregnadien-17α,21-diol-3,20-dione-21-acetate. In addition, dosage forms 40 and 42 may include a combination of a glucocorticoid and an angiostatic steroid as pharmaceutically active agents. For this combination, preferred glucocorticoids include dexamethasone, fluoromethalone, medrysone, betamethasone, triamcinolone, triamcinolone acetonide, prednisone, prednisolone, hydrocortisone, rimexolone, and pharmaceuitcally acceptable salts thereof, and preferred angiostatic steroids include 4,9(11)-Pregnadien-17α,21-diol-3,20-dione and 4,9(11)-Pregnadien-17α,21-diol-3,20-dione-21-acetate. Dosage forms 40 and 42 may also comprise conventional non-active excipients to enhance the stability, solubility, penetrability, or other properties of the active agent.

Device 10 is especially suitable for the delivery of a dosage form 40 and a dosage form 42 that exhibit some kind of mutual incompatibility and are best kept separate until just before delivery. In addition, dosage form 40 may include one of the ophthalmically acceptable pharmaceutically active agents suitable for localized delivery to the posterior segment of the eye mentioned hereinabove, and dosage form 42 may include a biocompatible polymer for preventing drug reflux during sub-Tenon, juxtascleral delivery of a drug depot to the posterior segment of the eye. A preferred polymer is a biocompatible, bioerodable polymer.

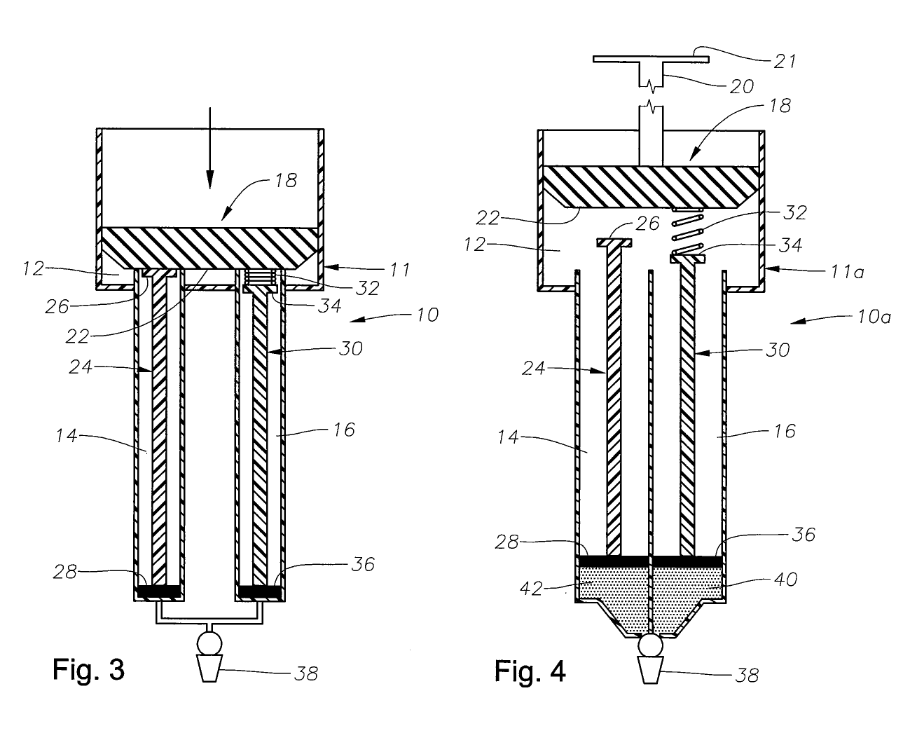

The following describes a preferred procedure by which a physician may use drug delivery device 10 for sub-Tenon, juxtascleral delivery of a drug depot to the posterior segment of an eye. Preferred cannulae for cannula 38 for such drug delivery are disclosed in U.S. Pat. No. 6,413,245. In the superior temporal quadrant of the eye, the physician uses fine scissors to create a small incision in the conjuctiva and Tenon's capsule to bare sclera at a point about 8 mm to about 9 mm posterior to the limbus. Cannula 38 of device 10 is then inserted through the incision. The distal tip of cannula 38 is advanced along the curvature of the sclera until the tip is located in the desired position. The physician then slowly depresses head 21 of handle 20 so that sealing member 22 of plunger assembly 18 cooperates with spring member 32 and contact member 34 of actuation assembly 30 to slide sealing member 36 toward cannula 38. As sealing member 36 is moved toward cannula 38, dosage form 40, which contains an appropriate pharmaceutically active agent, is slowly dispensed from cannula 38 to create a drug depot on the outer surface of the sclera below the Tenon's capsule. When sealing member 36 reaches the position shown in FIG. 2, spring member 32 is partially compressed, substantially all of dosage form 40 has been dispensed from cannula 38, and all of dosage form 42 remains in actuation chamber 14. The spring force of spring member 32 may be optimized for different volumes, forms, viscosities, and delivery rates of dosage form 40. As the physician continues to slowly depress head 21 of handle 20, sealing member 22 then cooperates with contact member 26 of actuation assembly 24 to slide sealing member 28 toward cannula 38. As sealing member 28 is moved toward cannula 38, dosage form 42, which contains a biocompatible, bioerodable polymer, is slowly dispensed from cannula 38 to seal the sub-Tenons space anterior to the drug depot and prevent reflux of dosage form 40. When sealing member 28 reaches the position shown in FIG. 3, spring member 32 is fully compressed, and substantially all of dosage form 42 has been dispensed from cannula 38. The physician slowly withdraws cannula 38 from the incision. The physician then applies an antibiotic ointment, and optionally applies a pressure patch to the incision.

As shown in FIG. 4, drug delivery device 10a has a substantially identical structure to device 10 with the exception that actuation chambers 14 and 16 are formed adjacent to one another instead of with a space therebetween like in device 10. The operation of device 10a is substantially identical to the operation of device 10.

From the above, it may be appreciated that the present invention provides an improved device for the administration of an ophthalmic drug, especially to the posterior segment of the eye. The device of the present invention also minimizes or prevents drug reflux during ophthalmic drug delivery. The device is safe for the patient, easy for the physician to use, and improves the efficacy of drug administration.

The present invention is illustrated herein by example, and various modifications may be made by a person of ordinary skill in the art. For example, although the use of the device of the present invention is described above in connection with sub-Tenon, juxtascleral delivery of a drug depot to the posterior segment, it can also be utilized in connection with other ophthalmic or non-ophthalmic drug delivery. As another example, handle 20 may be replaced with an automated assembly for displacing sealing member 22, if desired.

What is claimed is:

1. An ophthalmic drug delivery device, comprising:
a body having a plunger chamber, a first actuation chamber, and a second actuation chamber;
a plunger assembly having a first sealing member slidably disposed within said plunger chamber;
a first actuation assembly having a first contact member disposed in said plunger chamber, a second sealing member slidably disposed in said first actuation chamber, and a spring member disposed between said first sealing member and said first contact member;
a second actuation assembly having a second contact member disposed in said plunger chamber and a third sealing member slidably disposed in said second actuation chamber; and
a cannula fluidly coupled to said first actuation chamber and said second actuation chamber.

2. The ophthalmic drug delivery device of claim 1 further comprising:
a first dosage form disposed in said first actuation chamber between said second sealing member and said cannula; and
a second dosage form disposed in said second actuation chamber between said third sealing member and said cannula.

3. The ophthalmic drug delivery device of claim 2 wherein said spring member enables dispensing of said first dosage form from said cannula prior to dispensing of said second dosage form from said cannula.

4. The ophthalmic drug delivery device of claim 3 wherein:
said plunger assembly is coupled to a displacing member;
movement of said displacing member toward said cannula causes said plunger assembly, said spring member, and said first actuation assembly to dispense said first dosage form from said cannula; and
further movement of said displacing member toward said cannula causes said plunger assembly and said second actuation assembly to dispense said second dosage form from said cannula.

5. The ophthalmic drug delivery device of claim 4 wherein said displacing member is a handle.

6. The ophthalmic drug delivery device of claim 4 wherein said displacing member is an automated assembly for displacing said first sealing member.

7. The ophthalmic drug delivery device of claim 3 wherein said first dosage form is incompatible with said second dosage form.

8. The ophthalmic drug delivery device of claim 3 wherein:
said first dosage form comprises an ophthalmically acceptable pharmaceutically agent; and
said second dosage form is for preventing reflux of said first dosage form after dispensing into an eye.

9. The ophthalmic drug delivery device of claim 8 wherein said second dosage form comprises a biocompatible polymer for preventing reflux of said first dosage form after dispensing into an eye.

10. An ophthalmic drug delivery device, comprising:
a body having a first actuation chamber and a second actuation chamber;
a first actuation assembly having a first sealing member slidably disposed in said first actuation chamber;
a second actuation assembly having a second sealing member slidably disposed in said second actuation chamber;
a plunger assembly for actuating said first actuation assembly independently of said second actuation assembly;
a cannula fluidly coupled to said first actuation chamber and said second actuation chamber, said cannula comprising a distal portion having a radius of curvature substantially equal to a radius of curvature of a globe of a human eye;
a first dosage form disposed in said first actuation chamber between said first sealing member and said cannula, said first dosage form comprising an ophthalmically acceptable pharmaceutically active agent; and
a second dosage form disposed in said second actuation chamber between said second sealing member and said cannula, wherein said second dosage form is for preventing reflux of said first dosage form after dispensing into an eye.

11. The ophthalmic drug delivery device of claim 10 wherein said second dosage form comprises a biocompatible polymer for preventing reflux of said first dosage form after dispensing into an eye.
